(19) United States
(12) Patent Application Publication
(10) Pub. No.: US 2025/0375419 A1
(43) Pub. Date: Dec. 11, 2025

(54) NOVEL RAS INHIBITORS

(71) Applicant: KHR Biotec GmbH, Hamburg (DE)

(72) Inventor: Krishnaraj Rajalingam, Nieder-Olm (DE)

(21) Appl. No.: 18/874,233

(22) PCT Filed: Jun. 12, 2023
(86) PCT No.: PCT/EP2023/065615

§ 371 Date: Dec. 12, 2024

(30) Foreign Application Priority Data
Jun. 13, 2022 (EP) 22020278.2

(51) Int. Cl. A61K 31/4045; A61K 31/519; A61P 35/00

(52) U.S. Cl. CPC A61K 31/4045; A61K 31/519; A61P 35/00

(57) ABSTRACT

The present invention relates to the use of compounds of formula (I) as RAS inhibitors and as a medicament, in particular for use in treating proliferative disorders, inflammatory diseases and/or genetic disorders. The present invention relates further to a pharmaceutical composition comprising the compounds of formula (I). Moreover, the present invention relates to a method of inhibiting growth, proliferation or metastasis of cancer cells in a subject in need thereof, in particular which may encompass subsets of patients defined by their mutational status of the RAS oncogene or patients who might have developed resistance to the standard of care or treatment with RAS mutation specific inhibitors. The present invention also relates to a method of inhibiting RAS molecules in treating genetic disorders like RASopathies or inflammatory disorders like Adenomyosis where KRAS gene is mutationally activated. In addition, the present invention relates to a method of inhibiting proliferation and/or secretion of factors from a cell population sensitive towards inhibiting RAS activation in vitro, in particular sensitive towards inhibiting KRAS, HRAS and NRAS activation in vitro. Furthermore, the present invention relates to a kit containing a formulation comprising a pharmaceutical composition comprising a compound of formula (I).

The present invention relates to the use of compounds of formula (I) as RAS inhibitors and as a medicament, in particular for use in treating proliferative disorders, inflammatory diseases and/or genetic disorders. The present invention relates further to a pharmaceutical composition comprising the compounds of formula (I). Moreover, the present invention relates to a method of inhibiting growth, proliferation or metastasis of cancer cells in a subject in need thereof, in particular which may encompass subsets of patients defined by their mutational status of the RAS oncogene or patients who might have developed resistance to the standard of care or treatment with RAS mutation specific inhibitors. The present invention also relates to a method of inhibiting RAS molecules in treating genetic disorders like RASopathies or inflammatory disorders like Adenomyosis where KRAS gene is mutationally activated. In addition, the present invention relates to a method of inhibiting proliferation and/or secretion of factors from a cell population sensitive towards inhibiting RAS activation in vitro, in particular sensitive towards inhibiting KRAS, HRAS and NRAS activation in vitro. Furthermore, the present invention relates to a kit containing a formulation comprising a pharmaceutical composition comprising a compound of formula (I).

### BACKGROUND OF THE INVENTION

RAS proteins represent a group of closely related monomeric globular proteins which are associated with the plasma membrane and are able to bind either GDP or GTP. RAS that contains bound GDP represents the “inactive” state, whereas the binding of GTP to RAS in exchange to a GDP represents the “active” state, such that the protein is able to interact with other “effector” proteins of downstream targets. RAS proteins can be regarded as small GTPases that function as molecular switches controlling the transmission of extracellular signals from outside of the cell to the nucleus by various effector proteins. Activating RAS mutations are detected in inflammatory disorders like Adenomyosis/endometriosis which contributes to proliferation and invasions of endometrial cells and resistance to Progesterone (S. Inoue, Nature Comm. 2019, 10, 5785; PMID: 31857578).

There are three RAS isoforms (KRAS, HRAS and NRAS) and their activation cycle is regulated by the binding of GDP or GTP which in turn is controlled by GAPs or GEFs. In their GTP-bound form they bind to their effector proteins and trigger multiple signalling pathways that control various fundamental cellular processes.

Usually, mutations of RAS lead to defects in GAP-mediated GTP hydrolysis and thus result in the accumulation of RAS in the GTP-bound active state. This leads to uncontrollable proliferation, which is a hall mark of cancer cells. Uncontrolled activation of RAS is also detected in genetic disorders like RASOpathies.

Recent studies led to the development of mutation specific KRAS inhibitors that target the KRASG12C mutant which are approved for clinical use. Further inhibitors targeting the other mutant specific versions of KRAS are currently being developed.

Furthermore, it is known that patients frequently develop resistance to KRAS oncogene inhibitors, e.g. to KRAS G12 C inhibitors (Tanaka et al., Cancer Discov, 2021, PMID 33824136). In addition, the patients treated with KRAS G12 C inhibitors often develop secondary mutations in other RAS isoforms (Awad MM et al. New England J. Med., 2021 PMID34161704).

Tegaserod is used for the treatment of irritable bowel syndrome (IBS) with constipation. The effect results from activation of the 5-HT receptors (serotonin agonist).

Liu et al., J. Experimental & Clinical Cancer Research, 39 (1), 2020, relates to a study of tegaserod as an anti-cancer agent, its molecular mechanism and clinical implications.

Wu et al., Frontiers in Oncology, 11 (4), 2021, relates to a study of tegaserod maleate and its inhibition properties towards esophageal squamous cell carcinoma proliferation. Further, it is mentioned that tegaserod maleate inhibits the JAK/STAT3 signalling.

EP0505322 discloses compounds of general formula (I), in particular, tegaserod for the treatment of gastrointestinal disorders.

WO2020/211847relates to tegaserod and its use as JAK/STAT3 signalling pathway inhibitor and immunomodulator as well as its use as anti-tumor drug.

In contrast thereto, the compounds according to the present invention are more than 5-HT_{2B} receptor antagonist. The data here suggests that compounds uncouple active RAS from its effectors perhaps in the plane of the plasma membrane in cells. This event is much more proximal to the activation of the down stream kinase in a signaling cascade.

One cannot predict that if an effector molecule is inhibited by a compound e.g., tegaserod, activation of the three isoforms will also be inhibited.

None of the documents discloses or suggests that tegaserod is suitable for use as an inhibitor of RAS protein activation, in particular, wherein KRAS G12V, KRAS G12A, NRAS G12V, HRAS G12V, KRAS G12C, KRAS G12D, KRAS G12C/Y96D/S/C, KRAS G13C, KRAS G13D, KRASG13S, KRAS Q61H, KRAS Q61R or KRAS Q61K is involved.

Until now it was not known that tegaserod inhibits the expression and/or the activation of RAS. Activation is defined as ability of RAS to bind to its effector molecules like RAF kinases, PI3K kinases through the RAS binding domain (RBD) or RAS-Associated domain (RA domain) present in the effector proteins (like RASSF) in a GTP dependent manner. Further targeting the activation but not the stability can be advantageous in defined medical conditions while targeting both could be useful in combating certain subtypes of RAS mutated cancers.

Moreover, the mechanisms driving the activation of HRAS, NRAS, KRAS are different and each RAS isoform exhibits distinct functions and biological specificity. Thus, efforts are being made to target HRAS and NRAS in defined tumor subtypes where they function as an oncogenic driver.

Finally, targeting of other RAS isoforms like HRAS and NRAS is required to combat secondary, acquired resistance to the standard of care including several cancer therapeutics.

While KRASG12 specific C inhibitors have been developed, efforts to target HRAS and NRAS in cancers where these isoforms are mutated remains a challenge.

However, effective targeting, in particular inhibition of RAS oncogene activation of this RAS oncogenes with small molecules, in particular with limited toxicity is still a challenge.

It is therefore the object of the present invention to provide pharmaceutically active compounds that have the capability to inhibit the activation of RAS oncogenes, in particular in tumor cells at low concentrations with high specificity. This object is achieved by the use of compounds of formula (I). It was surprisingly found that the compounds of formula (I), in particular of formula (A) and (B) inhibit the activity of RAS oncogenes, in particular KRAS, HRAS and NRAS irrespective of their mutational status. Furthermore, it was surprisingly found that the compounds of formula (I), in particular, of formula (A) uncouples the active RAS-effector interaction in a GTP dependent manner (mutation agnostic). This was not predictable from prior art. Without being bound to any theory it is assumed that the interaction of mutationally activated RAS and its effector molecules is disrupted.

### SUMMARY OF THE INVENTION

The invention relates to compounds of formula (I)

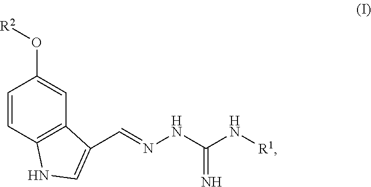

wherein
R¹ is C₁-C₆ alkyl;
R² is C₁-C₄ alkyl;
or a pharmaceutically acceptable salt thereof, for use as inhibitor of RAS protein activation, for treatment and/or prophylaxis of diseases, wherein RAS-signalling is involved.

In particular, the invention further relates to the compound of the formula (I)

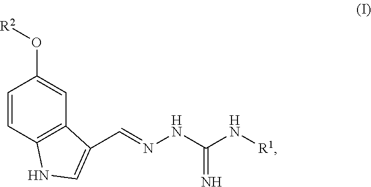

wherein
R¹ is C₁-C₆ alkyl;
R² is C₁-C₄ alkyl;
or a pharmaceutically acceptable salt thereof, for use as inhibitor of RAS protein activation for treatment and/or prophylaxis of proliferative disorders, wherein RAS-signaling is involved.

In particular, the invention further relates to the compound of the formula (I)

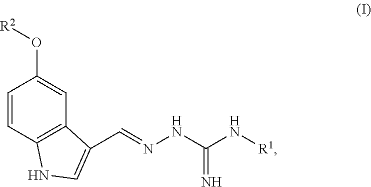

wherein
R¹ is C₁-C₆ alkyl;
R² is C₁-C₄ alkyl;
or a pharmaceutically acceptable salt thereof, for use as inhibitor of RAS protein activation for treatment and/or prophylaxis of genetic disorder, wherein RAS-signaling is involved, especially, for use as inhibitor of RAS protein activation for treatment and/or prophylaxis of RASophaties.

In particular, the invention further relates to the compound of the formula (I)

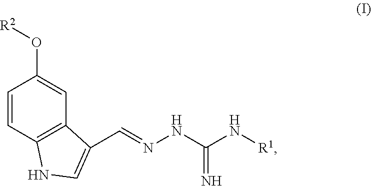

wherein
R¹ is C₁-C₆ alkyl;
R² is C₁-C₄ alkyl;
or a pharmaceutically acceptable salt thereof, for use as inhibitor of RAS protein activation for treatment and/or prophylaxis of inflammatory disorders, wherein RAS-signaling is involved, especially, for use as inhibitor of RAS protein activation for treatment and/or prophylaxis of endometriosis and/or adenomyosis, more especially, where KRAS is mutated.

In particular, the invention further relates to the compound of the formula (I)

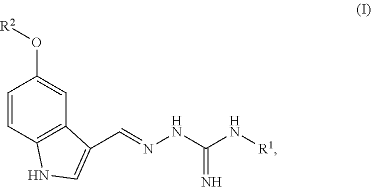

wherein
R¹ is C₁-C₆ alkyl;
R² is C₁-C₄ alkyl;
or a pharmaceutically acceptable salt thereof, as defined above and below, for use as inhibitor of RAS protein activation for treatment and/or prophylaxis of proliferative disorders and/or inflammatory diseases, wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved.

In particular, the invention further relates to the compound of the formula (I)

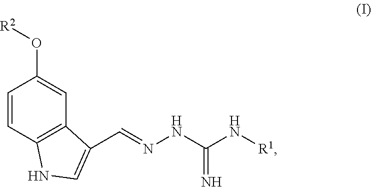

wherein
R¹ is C₁-C₆ alkyl;
R² is C₁-C₄ alkyl;
or a pharmaceutically acceptable salt thereof, as defined above and below, for use as inhibitor of RAS protein activation for treatment and/or prophylaxis of proliferative disorders and/or inflammatory diseases, which are resistant to treatment with RAS mutation specific inhibitors different from compounds of formula (I), preferably, wherein the resistance results from a secondary mutation, in particular results from a secondary mutation, wherein KRAS G12C/Y96D, KRAS G12C/Y96C and/or KRAS G12C/Y96S is involved.

The invention in particular relates to compounds of formula (I)

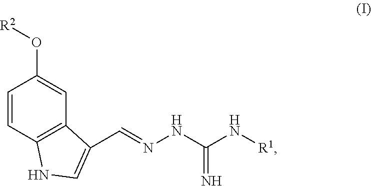

wherein
R¹ is C₁-C₆ alkyl;
R² is C₁-C₄ alkyl;
or a pharmaceutically acceptable salt thereof, for use as inhibitor of RAS protein activation.

The invention further relates to a composition (1) comprising at least one compound of formula (I) as defined above and below, in particular to compounds of formulae (A) and (B) as defined above and below, or a pharmaceutically acceptable salt, and RAS mutation specific inhibitors. The RAS mutation specific inhibitors are different from compounds of formula (I).

The invention further relates to compounds (I), in particular to compounds of formulae (A) and (B) as defined above and below, or a pharmaceutically acceptable salt thereof, or a composition (1) as defined above and below, or pharmaceutical compositions comprising at least one compound of formula (I), in particular selected from compounds (A) and (B) as defined above and below, or a composition (1) as defined above and below, for use as a RAS-RAF disruptor.

The invention further relates to compounds (I), in particular to compounds of formulae (A) and (B) as defined above and below, or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for use as a medicament.

The invention further relates to compounds (I) according to the invention, in particular to compounds of formulae (A) and/or (B) as defined above and below, or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for the treatment and/or prophylaxis of diseases.

The invention further relates to compounds (I) according to the invention, in particular to compounds of formulae (A) and/or (B) as defined above and below, or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for use in treating proliferative disorders.

The invention further relates to compounds (I) according to the invention, in particular to compounds of formulae (A) and/or (B) as defined above and below, or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for use in treating genetic disorders.

The invention further relates to compounds (I) according to the invention, in particular to compounds of formulae (A) and/or (B) as defined above and below, or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for use in treating RASophatie.

The invention further relates to compounds (I) according to the invention, in particular to compounds of formulae (A) and/or (B) as defined above and below, or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for use in treating inflammatory diseases.

The invention further relates to compounds (I) according to the invention, in particular to compounds of formulae (A) and/or (B) as defined above and below, or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for use in treating endometriosis and/or adenomyosis, more especially, where KRAS is mutated.

The invention further relates to compounds (I) according to the invention, in particular to compounds of formulae (A) and/or (B) as defined above and below or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for use in treating cancer.

The invention further relates to compounds (I) according to the invention, in particular to compounds of formulae (A) and/or (B) as defined above and below or a pharmaceutically acceptable salt thereof, or a composition (1) as defined above and below, or a pharmaceutical compositions comprising at least one compound of formula (I), in particular selected from compounds (A) and (B) as defined above or below, or a composition (1) as defined above and below, for use as inhibitor of RAS protein (KRAS, HRAS or NRAS oncogenes) activation.

The invention further relates to compounds (I) according to the invention, in particular to compounds of formulae (A) and/or (B) as defined above and below or a pharmaceutically acceptable salt thereof, or a composition (1) as defined above and below, or a pharmaceutical compositions comprising at least one compound of formula (I), in particular selected from compounds (A) and (B) as defined above or below or a composition (1) as defined above and below, for treating or preventing any diseases or conditions that are associated with the activity of RAS protein (RAS oncogene).

The invention further relates to compounds (I) according to the invention, in particular to compounds of formulae (A) and/or (B) as defined above and below or a pharmaceutically acceptable salt thereof, or a composition (1) as defined above and below, or a pharmaceutical compositions comprising at least one compound of formula (I), in particular selected from compounds (A) and (B) as defined above and below, or a composition (1) as defined above and below, for use in treating proliferative disorders, inflammatory diseases and/or genetic disorders, wherein RAS-signalling is involved, preferably wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved

The invention further relates to a pharmaceutical composition comprising at least one compound (I) according to the invention, in particular compounds of formulae (A) and/or (B) as defined above and below, or a pharmaceutically acceptable salt thereof, and a pharmaceutically acceptable carrier.

The invention further relates to a pharmaceutical composition as defined above and below, for use as inhibitor of RAS protein activation for treatment and/or prophylaxis of proliferative disorders and/or inflammatory diseases, which are resistant to treatment with RAS mutation specific inhibitors different from compounds of formula (I), preferably wherein the resistance results from a secondary mutation, in particular results from a secondary mutation, wherein KRAS G12C/Y96D, KRAS G12C/Y96C and/or KRAS G12C/Y96S is involved.

The invention further relates to a pharmaceutical composition comprising at least one composition (1) as defined above and below, and a pharmaceutically acceptable carrier.

The invention further relates to a pharmaceutical composition comprising at least one compound (I) according to the invention, in particular compound of formulae (A) and/or (B) as defined above and below, or a pharmaceutically acceptable salt thereof, or a composition (1) as defined above and below, wherein the pharmaceutical composition additionally comprises a further active substance, preferably selected from chemotherapeutic agents, radiotherapeutic agents, immuno-oncology agents and combinations thereof.

The invention further relates to a method of inhibiting growth, proliferation, or metastasis of cancer cells in a subject in need thereof, said method comprising administering to the subject a therapeutically effective amount of a compound (I) according to the invention, in particular a compound of formulae (A) and/or (B) as defined above and below, or a therapeutically acceptable salt thereof, or a composition (1) as defined above and below or a pharmaceutical composition comprising at least one compound of formula (I), in particular selected from compounds (A) and (B) as defined above and below, or a composition (1) as defined above and below.

The invention further relates to a method of inhibiting proliferation of a cell population sensitive towards inhibiting RAS activation in vitro or ex vivo, the method comprising contacting the cell population with a compound (I) according to the invention, in particular a compound of formulae (A) and/or (B) as defined above and below, or a therapeutically acceptable salt thereof, or a composition (1) as defined above and below, or a pharmaceutical compositions comprising at least one compound of formulae (I), in particular selected from compounds (A) and (B) as defined above and below, or a composition (1) as defined above and below.

The invention further relates to a kit containing a formulation comprising: a) a compound (I) according to the invention, in particular a compound of formulae (A) and/or (B) as defined above and below, or a composition (1) as defined above and below, or a pharmaceutical composition comprising a compound (I) according to the invention, in particular a compound of formulae (A) and/or (B) as defined above or below, or a therapeutically acceptable salt thereof, or a composition (1) as defined above and below, and a pharmaceutically acceptable carrier; and b) instructions for dosing of the pharmaceutical composition for the treatment of a disorder in which inhibition of RAS activation is effective in treating the disorder.

### DESCRIPTION OF THE INVENTION

The invention has the following advantages:
The compounds according to the invention exhibit advantageous RAS inhibition properties. In other words, the compounds according to the invention qualify as inhibitors of RAS oncogene activation. It is assumed that RAS-effector interaction especially when RAS is activated by oncogenic somatic mutations is disrupted.
The compounds inhibit KRAS irrespective of the mutations at concentrations which are pharmacologically achievable in human subjects. The tolerability of these molecules is well studied in the context of other disease entities like irritable bowel syndrome
The compounds inhibit NRAS and HRAS by functionally uncoupling their binding to their effectors in the cells.

### Compounds of Formula (I)

Inactivation in the sense of the invention means inhibiting the activity of a protein, in particular RAS protein, especially NRAS, KRAS or HRAS protein, based on direct interaction of at least one of the compounds of formula (I) and the proteins including prohibitions involved in complex with RAS proteins. This interaction is not a translation process or part of a translation process.

Further, activations mean the ability of RAS to bind to its effector molecules like RAF kinases, PI3K kinases through the RAS binding domain (RBD) or RAS-Associated domain (RA domain) present in the effector proteins (like RASSF) in a GTP dependent manner.

The term “synergistic” or “synergism” as used herein refers to a therapeutic combination which is more effective than the additive effects of the two or more single agents. That means the term “synergistic effect” refers to the effect for a given combination of two compounds where the activity of the combination exceeds the total of the individual activities of the compounds when applied separately. For this reason, the combination can, based on the individual components, be used at lower application rates to achieve a therapeutical effect comparable to the individual components.

The following equation is applied to determine whether the combination of compound of formula (I) and at least one further compound, different from compounds of formula (I), shows a synergistic effect:

$$E = X + Y - ( X \cdot Y / 100 ) ,$$

where X=effect in percent using compound of formula (I) at an application rate a;
Y=effect in percent using compound of formula (II) at application rate b;
E=expected effect (in %) of compound of formula (I)+compound of formula (II) at application rates a+b.

In the equation, the value E corresponds to the effect (inhibition) which is to be expected if the activity of the individual compounds is additive. If the observed effect is higher than the value E calculated according to the equation, a synergistic effect is present.

The term “proliferative disorders” refer to disorders that are associated with some degree of abnormal cell proliferation. Preferably, proliferative disorder is cancer. In particular, the cancer is selected from prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, haematological malignancies (inclooding blood, bone marrow and lymph nodes) or testicular seminoma.

Unless specifically stated otherwise herein, references made in the singular may also include the plural. For example, “a” and “an” may refer to either one, or one or more. In the context of the invention, the prefix Cₙ-Cₘ indicates the number of carbon atoms that a molecule or residue designated thereby may contain.

In the context of the invention, the expression C₁-C₄-alkyl refers to unbranched or branched saturated hydrocarbon groups having 1 to 4 carbon atoms. C₁-C₄-alkyl are e.g. methyl, ethyl, propyl, 1-methylethyl, butyl, 1-methylpropyl, 2-methylpropyl, 1,1-dimethylethyl.

The compounds of formulae (I), (A) and (B) form salts which are also within the scope of this invention. Pharmaceutically acceptable (i.e. non-toxic, physiologically acceptable) salts are preferred, although other salts are also useful, e.g., in isolation or purification steps which may be employed during preparation. Salts of the compounds of formulae (I), (A) and (B) may be formed, for example, by reacting a compound of formulae (I), (A) and (B) with at least one acid or base. The acid or base is added in an amount suitable for partial or complete neutralization e.g. an equivalent amount.

The phrase “pharmaceutically acceptable salt(s)” as used herein, unless otherwise indicated, includes salts containing pharmacologically acceptable anions or cations, such as the hydrochloride, hydrobromide, hydroiodide, nitrate, sulfate, bisulfate (hydrogen sulfate), phosphate, hydrogen phosphate, dihydrogen phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e. 4,4′-methylene-bis-(3-hydroxy-2-naphthoate)] salts.

Preferred are compounds of formula (I), wherein
R¹ is C₃-C₆ alkyl;
R² is C₁-C₂ alkyl;
or a pharmaceutically acceptable salt thereof, for use as inhibitor of RAS protein activation.

In particular R¹ is n-pentyl

R² is methyl.

In a first embodiment the compound of formula (I) is the compound A (also 1-{[(5-Methoxy-1H-indol-3-yl)methyliden]amino}-3-pentylguanidin, tegaserod, CAS no.: 145158-71-0)

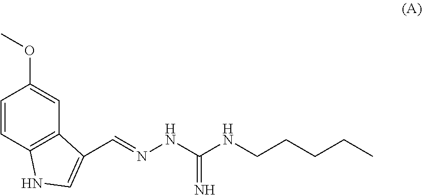 [SMILES: CCCCCNC(=N)N/N=C/c1c[nH]c2ccc(OC)cc12]

or a pharmaceutically acceptable salt thereof, for use as inhibitor of RAS protein activation.

Preferably, the invention relates to compound A or a pharmaceutically acceptable salt thereof, for use as inhibitor of RAS protein activation, for treatment and/or prophylaxis of diseases, wherein RAS-signaling is involved.

In particular, the invention relates to compound A or a pharmaceutically acceptable salt thereof, for use as inhibitor of RAS protein activation, for treatment and/or prophylaxis of proliferative disorders, inflammatory diseases and/or genetic disorders, wherein RAS-signaling is involved.

Especially, the invention relates to compound A or a pharmaceutically acceptable salt thereof, for use as inhibitor of RAS protein activation, for treatment and/or prophylaxis of proliferative disorders, inflammatory diseases and/or genetic disorders, wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D,

KRASG13S, KRAS Q61H, KRAS Q61R or KRAS Q61K is involved.

In another preferred embodiment, the invention relates to compound A or a pharmaceutically acceptable salt thereof, for use as inhibitor of RAS protein activation for treatment and/or prophylaxis of proliferative disorders and/or inflammatory diseases, which are resistant to treatment with RAS mutation specific inhibitors different from compounds of formula (I), preferably wherein the resistance results from a secondary mutation, in particular results from a secondary mutation, wherein KRAS G12C/Y96D, KRAS G12C/Y96C and/or KRAS G12C/Y96S is involved.

In a second embodiment the compound of formula (I) is the compound B (also 12-[(5-Methoxy-1H-indol-3-yl) methylene]-N-pentyl-hydrazinecarboximidamide maleate, tegaserod maleate, CAS no.: 189188-57-6)

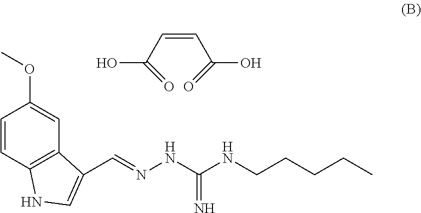 [SMILES: CCCCCNC(=N)N/N=C/c1c[nH]c2ccc(OC)cc12 ; O=C(O)/C=C\C(=O)O]

or a pharmaceutically acceptable salt thereof, for use as inhibitor of RAS protein activation.

Preferably, the invention relates to compound B or a pharmaceutically acceptable salt thereof, for use as inhibitor of RAS protein activation, for treatment and/or prophylaxis of diseases, wherein RAS-signaling is involved.

In particular, the invention relates to compound B or a pharmaceutically acceptable salt thereof, for use as inhibitor of RAS protein activation, for treatment and/or prophylaxis of proliferative disorders, inflammatory diseases and/or genetic disorders, wherein RAS-signaling is involved.

Especially, the invention relates to compound B or a pharmaceutically acceptable salt thereof, for use as inhibitor of RAS protein activation, for treatment and/or prophylaxis of proliferative disorders, inflammatory diseases and/or genetic disorders, wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved.

In another preferred embodiment, the invention relates to compound B or a pharmaceutically acceptable salt thereof, for use as inhibitor of RAS protein activation for treatment and/or prophylaxis of proliferative disorders and/or inflammatory diseases, which are resistant to treatment with RAS mutation specific inhibitors different from compounds of formula (I), preferably wherein the resistance results from a secondary mutation, in particular results from a secondary mutation, wherein KRAS G12C/Y96D is involved.

A further aspect of the invention is a composition (1) comprising at least one compound of formula (I) as defined above and below, or a pharmaceutically acceptable salt thereof, and at least one RAS mutation specific inhibitor.

In one embodiment composition (1) comprising compound (A) or a pharmaceutically acceptable salt thereof, and at least one RAS mutation specific inhibitor, in particular KRAS mutation specific inhibitors, especially sotorasib (also known as AMG510 or (1M)-6-Fluor-7-(2-fluor-6-hydroxyphenyl)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-on) and/or adagrasib (also known as MRTX848, or (2S)-4-[7-(8-Chlor-1-naphthyl)-5,6,7,8-tetrahydro-2-[[(2″S″)-1-methyl-2-pyrrolidinyl]methoxy]pyrido [3,4-“d”]pyrimidin-4-yl]-1-(2-fluor-1-oxo-2-propen-1-yl)-2-piperazinacetonitril)).

In second embodiment composition (1) comprising compound (B) or a pharmaceutically acceptable salt thereof, and at least one RAS mutation specific inhibitor, in particular KRAS mutation specific inhibitors, especially sotorasib and/or adagrasib.

### Pharmaceutical Composition

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The phrase “therapeutically effective” is intended to qualify the amount of each agent, which will achieve the goal of improvement in disorder severity and the frequency of incidence, while avoiding adverse side-effects typically associated with alternative therapies. For example, effective anticancer agents prolong the survivability of the patient or his/her life quality, inhibit the rapidly proliferating cell growth associated with the formation of neoplasms, or effect a regression of the neoplasms.

The terms “treat,” “treating,” and “treatment,” as used herein, refer to any type of intervention or process performed on, or administering an active agent to, the subject with the objective of reversing, alleviating, ameliorating, inhibiting or slowing down or preventing the progression, development, severity or recurrence of a symptom, complication, condition or biochemical indicia associated with a disease. By contrast, “prophylaxis” or “prevention” refers to administration to a subject who does not have a disease to prevent the disease from occurring.

As used herein, the term “cell” is meant to refer to a cell that is in vitro, ex vivo or in vivo. In the sense of the invention, an ex vivo cell can be part of a tissue sample excised from an organism such as a mammal. In the sense of the invention, an in vitro cell can be a cell in a cell culture. In the sense of the invention, an in vivo cell is a cell living in an organism such as a mammal.

The term “patient” includes humans and animals that receive either therapeutic or prophylactic treatment.

The term “subject” include

s any human or animal. For example, the methods and compositions herein disclosed can be used to treat a subject having cancer.

A (non-human) animal includes all vertebrates, e.g. mammals and non-mammals, including cows, sheep, pigs, goats, horses, poultry, dogs, cats, non-human primates, rodents etc. In one embodiment, the subject is a human subject.

The phrase “pharmaceutically acceptable carrier” as used herein means a pharmaceutically acceptable material, composition or vehicle, such as a liquid or solid diluent, solvent, excipient, manufacturing aid (e.g. lubricant) or encapsulating material, involved in carrying or transporting the subject compound from one organ, or portion of the body, to another organ, or portion of the body. Each carrier must be “acceptable” in the sense of being compatible with the other ingredients of the formulation.

Suitable other ingredients are the afore-mentioned carrier and further additives, including adjuvants, preserving agents, fillers, flow regulating agents, disintegrating agents, wetting agents, emulsifying agents, suspending agents, sweetening agents, flavoring agents, bittering agents, perfuming agents, antibacterial agents, antifungal agents, lubricating agents, dispensing agents, etc. Suitable additives are selected depending on the nature of the mode of administration and dosage forms; and not injurious to the patient.

The term “pharmaceutical composition” means a composition comprising a compound of the invention in combination with at least one further compound selected from
a) at least one further pharmaceutically active substance and
b) at least one additional pharmaceutically acceptable carrier and or additive.

The term “RAS inhibitor” refers to an agent capable of decreasing RAS protein levels, decreasing RAS activity levels and/or inhibiting RAS expression levels in the cells. The RAS inhibitor may be a reversible or irreversible inhibitor. As used herein, “RAS” protein refers to a protein that is a member of a family of related proteins that are expressed in all human and animal cell lineages and organs. All RAS protein family members belong to a class of proteins called small GTPase (also known as small G proteins, a family of hydrolase enzymes that can bind and hydrolyse GTP), and are involved in transmitting signals within cells (cellular signal transduction). RAS is the prototypical member of the RAS superfamily of proteins, which are all related in three-dimensional structure and regulate diverse cell behaviours. When RAS is ‘switched on’ by incoming signals, it subsequently switches on other proteins, which ultimately turn on genes involved in cell growth, differentiation, and survival. Mutations in RAS genes can lead to the production of permanently activated RAS proteins, which can cause unintended and overactive signaling inside the cell, even in the absence of incoming signals. Because these signals result in cell growth and division, overactive RAS signaling can ultimately lead to cancer. The three RAS genes in humans (HRAS, KRAS, and NRAS) are the most common oncogenes in human cancer. As mentioned, the clinically most notable members of the RAS subfamily are HRAS, KRAS and NRAS. However, there are other members of this subfamily, which are e.g. selected from DIRAS1, DIRAS2, DIRAS3, ERAS, GEM, MRAS, NKIRAS1, NKIRAS2, NRAS, RALA, RALB, RAP1A, RAP1B, RAP2A, RAP2B, RAP2C, RASD1, RASD2, RASL10A, RASL10B, RASL11A, RASL11B, RASL12, REM1, REM2, RERG, RERGL, RRAD, RRAS, RRAS2. In other words, the most common alterations in NRAS are NRAS Mutation (2.87%), NRAS Exon 3 Mutation (1.90%), NRAS Exon 3 Missense (1.88%), NRAS Codon 61 Missense (1.72%), and NRAS Exon 2 Mutation (0.95%) The most common alterations in HRAS are HRAS Mutation (0.77%), HRAS Missense (0.75%), HRAS Exon 2 Mutation (0.30%), HRAS Codon 61 Missense (0.26%), and HRAS Q61R (0.14%) The most common alterations in HRAS are HRAS Mutation (0.77%), HRAS Missense (0.75%), HRAS Exon 2 Mutation (0.30%), HRAS Codon 61 Missense (0.26%), and HRAS Q61R (0.14%) (Source Mycancer genome portal).

The compound(s) of formulae (I), (A) and (B) as defined above and the composition (1) as defined above and a pharmaceutical composition comprising at least one compound of formulae (I), (A) and (B) as defined above, or the composition (1) as defined above may be administered to humans and animals, preferably humans.

In principle any method of administration may be used to deliver the compound or pharmaceutical composition according to the invention to a subject. Suitable methods of administration are orally, enterally, parenterally, intravenously, topically, intramuscular, subcutaneous routes.

The compound(s) of formulae (I), (A) and (B) as defined above can selectively decrease RAS protein levels, decrease RAS activity levels, in particular decrease the activity levels of HRAS and NRAS, especially KRAS4A, and KRAS4B) in the cells. For example, the compound(s) of formulae (I), (A) and (B) as defined above can be used to selectively decrease RAS activity levels in cells or in an individual in need of a decrease in RAS protein levels, decrease in RAS activity levels by administering an inhibiting amount of compound(s) of formulae (I), (A) and (B) as defined above or a salt thereof.

In one embodiment, the present invention provides a combined preparation of a compound or compounds of formulae (I), (A) and (B) as defined above, and/or a pharmaceutically acceptable salt thereof, and (an) additional therapeutic agent(s) for simultaneous, separate or sequential use in the treatment and/or prophylaxis of (multiple) diseases, preferably of proliferative disorders (e.g. cancer), in particular disorders associated with the activity of RAS protein.

Additional therapeutic agent(s) are selected from chemotherapeutic agents, radiotherapeutic agents, immuno-oncology agents, and combinations thereof.

In one aspect, the compound(s) of formulae (I), (A) and (B) as defined above are sequentially administered prior to administration of the immuno-oncology agent. In another aspect, compound(s) of formulae (I), (A) and (B) as defined above are administered concurrently with the immuno-oncology agent. In yet another aspect, compound(s) of formulae (I), (A) and (B) as defined above are sequentially administered after administration of the immuno-oncology agent.

In another aspect, compound(s) of formulae (I), (A) and (B) as defined above may be co-formulated with an immuno-oncology agent.

Immuno-oncology agents include, for example, a small molecule drug, antibody or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Suitable of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1(PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTpR, LIGHT, DcR3, HVEM, VEGETL1A, TRAMP/DR3, EDAR, EDA1, XEDAR, EDA2, TNFR1, Lymphotoxin a/TNFp, TNFR2, TNFa, LTpR, Lymphotoxin a 1b2, FAS, FASL, RELT, DR6, TROY, NGFR.

In one aspect, T cell responses can be stimulated by a combination of compound(s) of formulae (I), (A) and (B) as defined above and one or more of:
(i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and
(ii) an agonist of a protein that stimulates T cell activation such as B7-1, B7-2, CD28, 4-1BB (CD 137), 4-1BBL, ICOS, ICOS-L, 0X40, OX40L, GITR, GITRL, CD70, CD27, CD40, DR3 and CD28H.

Other agents that can be combined with compound(s) of formulae (I), (A) and (B) as defined above for the treatment of cancer include antagonists of inhibitory receptors on NK cells or agonists of activating receptors on NK cells. For example, compound(s) of formulae (I), (A) and (B) as defined above can be combined with antagonists of KIR, such as Lirilumab.

Yet other agents for combination therapies include agents that inhibit or deplete macrophages or monocytes, including but not limited to CSF-1R antagonists such as CSF-1R antagonist antibodies including RG7155.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy can also embrace the administration of the therapeutic agents as described above in further combination with other biologically active ingredients and non-drug therapies (e.g surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Types of cancers that may be treated with the compounds of formulae (I), (A) and (B) as defined above include, but are not limited to, prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, haematological malignancies (including blood, bone marrow and lymph nodes) or testicular seminoma.

In one embodiment the invention relats to the inhibition of HRAS mutations, which are detected in bladder urothelial carcinoma, breast invasive ductal carcinoma, lung adenocarcinoma, prostatecarcinoma and colon adenocarcinoma. This accounts to nearly 0.94% of all human cancers and nearly 1.02% of solid tumours. In another embodiment the invention relats to the inhibition of HRAS mutations, which are also detected in other cancers selected from chronic myelomonocytic leukemia, non-nodgkin lymphoma, thyroid gland carcinoma, head and neck squamous cell carcinoma, squamous cell lung carcinoma, ovarian carcinoma, poorly differentiated thyroid gland carcinoma, squamous cell carcinoma, small cell lung carcinoma, glioma, low grade glioma, pancreatic carcinoma, acute lymphoblastic leukemia, histiocytic and dendritic cell neoplasm, multiple myeloma, neurofibromatosis type, pancreatic ductal adenocarcinoma, thyroid gland follicular carcinoma, embryonal rhabdomyosarcoma, malignant thyroid gland neoplasm, thyroid gland undifferentiated (anaplastic) carcinoma, thymic carcinoma, urothelial carcinoma, thyroid gland papillary carcinoma cutaneous melanoma, mucosal melanoma, endometrial carcinoma, malignant peripheral nerve sheath tumor, neuroblastoma, prostate carcinoma, soft tissue sarcoma, breast carcinoma, colorectal adenocarcinoma, gastric carcinoma, diffuse, large b-cell lymphoma, diffuse gliom, myeloid dysplastic syndrome, renal cell carcinoma, astrocytic tumor, hepatocellular carcinoma and shwannoma.

NRAS mutations are detected in nearly 3.03% of all human cancers with frequent mutations cutaneous melanoma, melanoma, colonadenocarcinoma, aml, thyroid carcinoma and lung adenocarcinoma. Therefore another embodiment of the invention relats to the inhibition of NRAS mutations detected in cutaneous melanoma, melanoma, colonadenocarcinoma, aml, thyroid carcinoma and lung adenocarcinoma. This accounts for nearly 2.83% of malignant solid tumour patients. In another embodiment the invention relats to the inhibition of NRAS mutations, which are also detected in other cancers selected from colorectal carcinoma, non-small cell lung carcinoma, acute myeloid leukemia, myelodysplastic syndromes, chronic myelomonocytic leukemia, colorectal adenocarcinoma, multiple myeloma, non-hodgkin lymphoma, pancreatic carcinoma, cutaneous melanoma, ovarian carcinoma, pancreatic ductal adenocarcinoma, acute lymphoblastic leukemia, thyroid gland carcinoma, glioma, neurofibromatosis type 1, poorly differentiated thyroid gland carcinoma, secondary acute myeloid leukemia, therapy-related acute myeloid leukemia, myelodysplastic syndrome with excess blasts-2, juvenile myelomonocytic leukemia, histiocytic and dendritic cell neoplasm, head and neck squamous cell carcinoma,small cell lung carcinoma, low grade glioma, squamous cell lung carcinoma, breast carcinoma, chronic myelomonocytic leukemia-2, chronic myelomonocytic leukemia, thyroid gland undifferentiated (anaplastic) carcinoma, embryonal rhabdomyosarcoma, thyroid gland follicular carcinoma, t-cell acute lymphoblastic leukemia, mucosal melanoma, chronic myelomonocytic leukemia-1,low grade ovarian serous adenocarcinoma,thyroid gland papillary carcinoma, refractory anemia with excess blasts, myeloid neoplasm myelodysplastic/myeloproliferative neoplasm, unclassifiable, rectal carcinoma, colon carcinoma, malignant peripheral nerve sheath tumor, cholangiocarcinoma, endometrial carcinoma mantle cell lymphoma, secondary myelodysplastic syndrome, therapy-related myelodysplastic syndrome, lymphoma neuronal and mixed neuronal-glial tumors, ganglioglioma, soft tissue sarcoma, bladder carcinoma, esophageal carcinom, sarcoma, thymic carcinoma, lung adenocarcinoma, lung carcinoma, uveal melanoma head and neck carcinoma, diffuse glioma, squamous cell carcinoma, chronic myeloid leukemia, adenocarcinoma of the gastroesophageal junction, glioblastoma neuroblastoma, astrocytic tumo, hepatocellular carcinoma, pancreatic adenocarcinomadiffuse large b-cell lymphoma, anaplastic astrocytoma, gastric adenocarcinoma, gastric carcinoma, prostate carcinoma, renal cell carcinoma, acute myeloid leukemia arising from previous myelodysplastic syndrome, b-cell acute lymphoblastic leukemia, double-hit lymphoma, dysembryoplastic neuroepithelial tumor, gangliocytoma, low-grade neuroepithelial tumor, peripheral t-cell lymphoma, mpilocytic astrocytoma, pilomyxoid astrocytoma, rhabdoid tumor and schwannoma.

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors can be optionally used in combination with the compound(s) of formulae (I), (A) and (B), as defined above, for treatment of RAS protein associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compound(s) of formulae (I), (A) and (B), as defined above, include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOY™. Compound(s) of formulae (I), (A) and (B) as defined above may also be combined with immunotherapy drugs, including cytokines such as interferon alpha, interleukin 2, and tumor necrosis factor (TNF) in the treatment of melanoma. Compound(s) of formulae (I), (A) and (B) as defined above may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compound(s) of formulae (I), (A) and (B) as defined, using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 38.9° C. to 40° C. Melphalan is the drug most often used in this chemotherapy procedure. This can be given with another agent called tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-a), etoposide, and teniposide.

Other cytotoxic agents include navelbene, CPT-11, anastrazole, letrazole, capecitabine, reloxafme, and droloxafme.

Also suitable are cytotoxic agents such as epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (HERCEPTIN®), antibodies to costimulatory molecules such as CTLA-4, 4-1BB and PD-1, or antibodies to cytokines (IL-IO or TGF-b).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

In a specific embodiment of the present invention, at least one compound of formulae (I), (A) and (B) as defined above and at least one chemotherapeutic agent are administered to the patient concurrently or sequentially. In other words, at least one compound of formulae (I), (A) and (B) as defined above may be administered first, at least one chemotherapeutic agent may be administered first, or at least one compound of formulae (I), (A) and (B) as defined above may be administered at the same time. Additionally, when more than one compound of formulae (I), (A) and (B) as defined above and/or chemotherapeutic agent is used, the compounds may be administered in any order.

The invention also provides pharmaceutically compositions which comprise a therapeutically effective amount of one or more of the compound(s) of formulae (I), (A) and (B), as defined above, formulated together with one or more pharmaceutically acceptable carriers (additives) and/or diluents, and optionally one or more additional therapeutic agents as described above.

The compound(s) of formulae (I), (A) and (B), as defined above, may be administered by any suitable route, preferably in the form of a pharmaceutical composition adapted to such a route, and in a dose effective for the treatment intended. The compound(s) and compositions of the compound(s) of formulae (I), (A) and (B) as defined above, can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g. as sterile injectable aqueous or non-aqueous solutions or suspensions); nasally, including administration to the nasal membranes, such as by inhalation spray; topically, such as in the form of a cream or ointment; or rectally, such as in the form of suppositories. They can be administered alone, but generally will be administered with a pharmaceutical carrier selected on the basis of the chosen route of administration and standard pharmaceutical practice.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or animal may vary widely depending on the condition of the patient and other factors, but, can be determined using routine methods.

Any pharmaceutical composition contemplated herein can, for example, be delivered orally via any acceptable and suitable oral preparation. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, bittering agents, coloring agents, demulcents, antioxidants, and preserving agents.

A tablet can, for example, be prepared by admixing at least one compound of formulae (I), (A) and (B), as defined above, and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasantly tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of formulae (I), (A) and (B) as defined above, and/or at least one salt thereof with at least one inert solid diluent, such as, for example, calcium carbonate; calcium phosphate; and kaolin.

Soft gelatin capsules can, for example, be prepared by mixing at least one compound of formulae (I), (A) and (B) as defined above, and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of formulae (I), (A) and (B) as defined above and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, hydroxypropyl-methylcellulose and hydroxypropyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of formulae (I), (A) and (B) as defined above and/or at least one pharmaceutically acceptable salt thereof in either a vegetable oil, such as, for example, arachis oil, olive oil, sesame oil and coconut oil or in mineral oil, such as, for example, liquid paraffin. An oily suspension can also contain at least one thickening agent, such as, for example, beeswax, hard paraffin and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of formulae (I), (A) and (B) as defined above and/or at least one pharmaceutically acceptable salt thereof with at least one dispersing and/or wetting agent; at least one suspending agent; and/or at least one preservative. Suitable dispersing agents, wetting agents, and suspending agents are as already described above.

Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of formulae (I), (A) and (B) as defined above and/or at least one pharmaceutically acceptable salt thereof can, for example, be prepared as an oil-in-water emulsion. The oily phase of the emulsions comprising compound(s) of formulae (I), (A) and (B) as defined above may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilize) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compound(s) of formulae (I), (A) and (B) as defined above and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers, including saline, dextrose, water or with cyclodextrin solubilization (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono-or diglycerides. In addition, fatty acids such as oleic acid find use in the preparation of injectables.

A sterile injectable oil-in-water microemulsion can, for example, be prepared by 1) dissolving at least one compound of formulae (I), (A) and (B) as defined above in an oily phase, such as, for example, a mixture of soybean oil and lecithin; 2) combining the compound(s) of formulae (I), (A) and (B) as defined above containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V. Jr. et al. Remington: The Science and Practice of Pharmacy (2 Volumes), 22nd Edition (2012), Pharmaceutical Press.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol poly ethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

The amounts of compounds that are administered and the dosage regimen for treating a disease condition with the compounds and/or compositions of this invention depend on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

Pharmaceutical compositions of this invention comprise at least one compound of formulae (I), (A) and (B) as defined above and/or at least one pharmaceutically acceptable salt thereof, or at least one composition (1) as defined above, and optionally an additional agent selected from any pharmaceutically acceptable carrier, adjuvant, and vehicle. Alternate compositions of this invention comprise a compound of the formulae (I), (A) and (B) as defined above, or a prodrug thereof, and a pharmaceutically acceptable carrier, adjuvant, or vehicle.

The present invention also includes pharmaceutical kits useful, for example, in the treatment or prevention of RAS protein-associated diseases. Thus, the present invention also relates to a kit containing a formulation comprising: a) a pharmaceutical composition comprising a compound of formulae (I), (A) and (B) as defined above, or a therapeutically acceptable salt thereof or at least one composition (1) as defined above and a pharmaceutically acceptable carrier; and b) instructions for dosing of the pharmaceutical composition for the treatment of a disorder in which inhibition of RAS activation is effective in treating the disorder.

Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compound(s) of the formulae (I), (A) and (B) may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g. oral tablets, capsules, elixirs, and syrups, and consistent with conventional pharmaceutical practices.

Dosage forms (pharmaceutical compositions) suitable for administration may contain from about 1 milligram to about 200 milligrams of active ingredient per dosage unit. In these pharmaceutical compositions the active ingredient will ordinarily be present in an amount of about 0.1-95% by weight based on the total weight of the composition.

A typical capsule for oral administration contains at least one of the compound of the formulae (I), (A) and (B) (250 mg), lactose (75 mg), and magnesium stearate (15 mg). The mixture is passed through a 60 mesh sieve and packed into a no. 1 gelatin capsule.

A typical injectable preparation is produced by aseptically placing at least one of the compound of the formulae (I), (A) and (B) (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compound of formulae (I), (A) and (B) as defined above, alone or in combination with a pharmaceutical carrier. Optionally, compound(s) of formulae (I), (A) and (B) as defined above can be used alone, in combination with other compound(s) of formulae (I), (A) and (B) as defined above, or in combination with one or more other therapeutic agent(s), e.g.

an anticancer agent or other pharmaceutically active material.

Regardless of the route of administration selected, the compound(s) of formulae (I), (A) and (B) as defined above, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound(s) of formulae (I), (A) and (B) as defined above employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of compound(s) of formulae (I), (A) and (B) as defined above employed in the pharmaceutical composition at levels lower than that required in order to achieve the desired therapeutic effect and gradually increase the dosage until the desired effect is achieved.

In general, a suitable daily dose of compound(s) of formulae (I), (A) and (B) as defined above will be that amount of the compound which is the lowest dose effective to produce a therapeutic effect. Such an effective dose will generally depend upon the factors described above. Generally, oral, intravenous, intracerebroventricular and subcutaneous doses of the compound(s) of formulae (I), (A) and (B) as defined above for a patient will range from about 0.01 to about 50 mg per kilogram of body weight per day.

If desired, the effective daily dose of the active compound may be administered as one, two, three, four, five, six or more sub-doses administered separately at appropriate intervals throughout the day, optionally, in unit dosage forms. In certain aspects of the invention, dosing is one administration per day.

While it is possible for compound(s) of formulae (I), (A) and (B) as defined above to be administered alone, it is preferable to administer the compound as a pharmaceutical formulation (composition).

The above other therapeutic agents, when employed in combination with the compound(s) of formulae (I), (A) and (B) as defined above, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

The invention will be illustrated further with reference to the examples that follow, without restricting the scope to the specific embodiments described. The invention includes all combinations of described and especially of preferred features that do not exclude each other.

The invention will be illustrated further with reference to the examples that follow, without restricting the scope to the specific embodiments described. The invention includes all combinations of described and especially of preferred features that do not exclude each other.

### DESCRIPTION OF THE DRAWINGS

FIGS. 1a) and 1b): NanoBit assay for RAS activation (KRAS G12V, NRAS G12V, HRAS G12V)

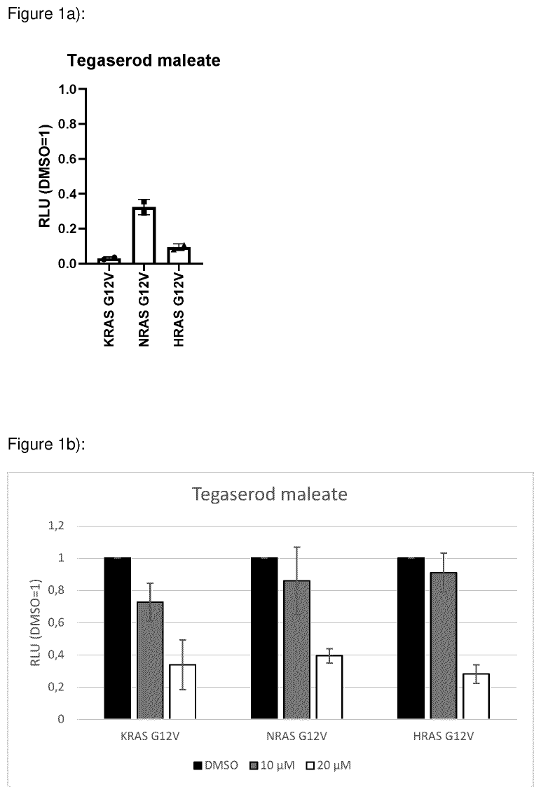

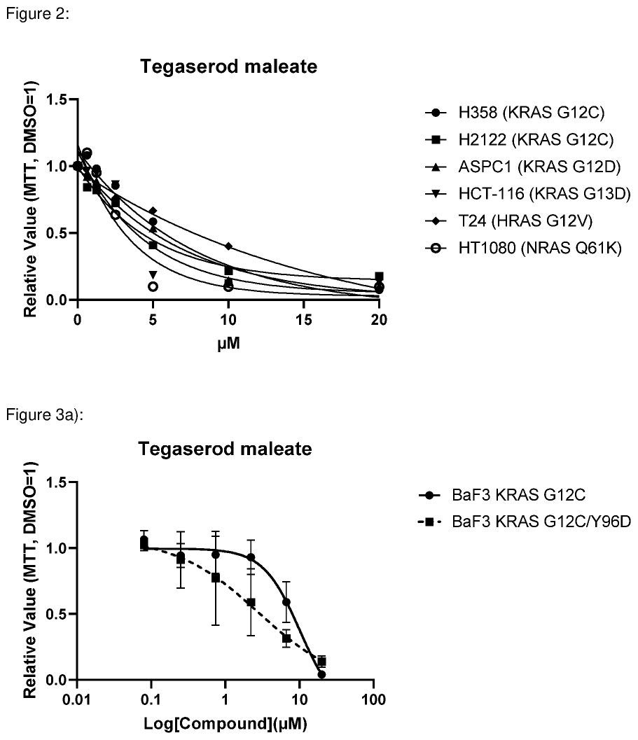

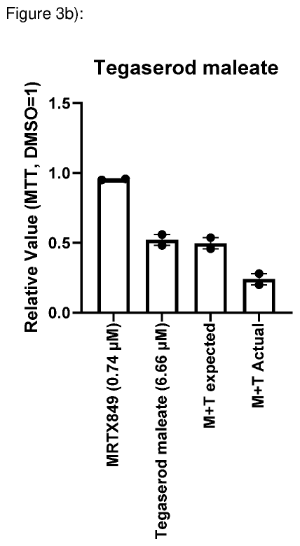

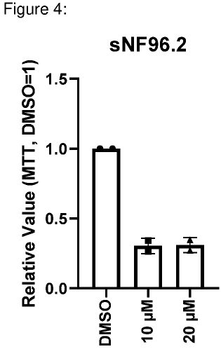

N-terminal LgBiT and C-terminal SmBiT construct was purchased from Promega and K, N and HRAS G12V (full length) was cloned with Xho I and Bgl II to LgBit and CRAF Ras binding domain (1-149) was cloned with EcoRI and Bgl II to SmBit. For transfections, 1 μg or 2 μg of (12 well/6 well) plasmids were transfected into cells with 0.5 mM of PEI reagent in 100 μl or 200 μl PBS. One day after transfection, cells were harvested and seeded into 96 well white plates, half area (Greiner). After an additional day the medium was changed to serum free DMEM and the cells were incubated for 2 h with inhibitor. NanoGlo assay was performed according to the manufacturer's instructions. The luminescence was measured using Tecan infinite (Tecan).

Tegaserode maleate K-RAS, N-RAS and H-RAS.

FIG. 2): MTT assay for cell growth

Metabolic activity was quantified using Cell Proliferation Kit I (Roche, Basel, Switzerland). Cells were seeded in 96-well cell culture plates, using 5000 cells per well for NCI-H358 cells. After treatment, 10 μl of MTT solution was added and incubated for 2 h in CO₂ incubator. Then 100 μl of solubilization buffer was added to each well and incubated overnight in CO₂ incubator. Cell viability, assessed by the amount of metabolized MTT, was quantified by measuring absorbance at 570 nm.

MTT assay for the growth of different tumour cell lines with RAS mutations in soft agar with Tegaserode maleate.

NCI-H358, H358, H2122, ASPC-1, HCT-116, T24 and HT1080 cells were used for the soft agar colony formation assay. After the treatment with Tegaserode maleate, the colonies were stained with MTT and the value was quantified by measuring absorbance at 570 nm after solubilization. The results represent mean±SEM from 3 independent experiments.

| | NCI-H358 | NCI-H2122 | ASPC1 | HCT-116 | T24 | HT1080 |
|---|---|---|---|---|---|---|
| IC50 | 5.39 | 3.37 | 4.76 | 3.12 | 11.31 | 2.43 |
| (μM) | | | | | | |

FIGS. 3a) und 3b): MTT assay for cell viability in 96 well cell culture plate Cells were treated with compounds for 48 h and cell viability was evaluated by MTT assay. The results shown are mean from 3 independent experiments. DMSO-treated cells were set as 1. The results shown are mean±SEM from 1-3 independent experiments.

FIG. 3a): Tegaserode maleate inhibits KRAS dependent cells irrespective of the presence of mutation. G12C/Y96D mutation is normally seen in patients, who develop resistance to G12C inhibitor. Thus, patients, who are resistant to G12C inhibitors can still be treated with Tegaserode maleate.

| | BaF3 KRAS | BaF3 KRAS |
|---|---|---|
| | G12C | G12C/Y96D |
| IC50 (μM) | 10.19 | 2.86 |

FIG. 4b): Comparison of the single compounds Tegaserode maleate and MRTX849 (Adagrasib) and the mixture of Tegaserode maleate (T) MRTX849 (M) in MTT assay in RAS mutated cells. Further, the calculated/expected values of the mixture and the actual/real values of the mixture is shown. Expected values were obtained using the equation:

$$Expected value = T + M - ( T \cdot M / 100 ) \\ Expected value = [ 100 - [ T + M - ( T \cdot M / 100 ) ] ] / 100 ( standardized )$$

FIG. 4: RASOpathie-Model of Tegaserode maleate; MTT assay for cell viability in 96 well cell culture plate

Cells were treated with Tegaserode maleate for 48 h and cell viability was evaluated by MTT assay. DMSO-treated cells were set as 1. The results shown are mean±SEM from 3 independent experiments.

Tegaserode maleate inhibits RAS protein of RASOpathie.

### CELL CULTURE

HeLa S3 (DSMZ) were authenticated by Eurofin genomics and cultured in DMEM (10% heat inactivated FBS). NCI-H358 cells were cultured in RPMI-1640 (10% heat inactivated FBS).

### EXAMPLES

Tegaserod maleate: 2-[(5-Methoxy-1H-indol-3-yl)methylene]-N-pentyl-hydrazinecarboximidamide maleate, CAS: 189188-57-6

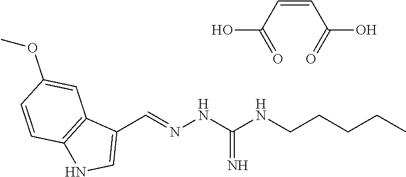 [SMILES: CCCCCNC(=N)N/N=C/c1c[nH]c2ccc(OC)cc12 ; O=C(O)/C=C\C(=O)O]

is commercial available by Sigma-Aldrich/Merck.

1. A compound of the formula (I) 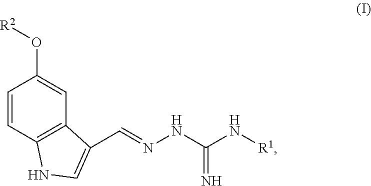
wherein
R¹ is C₁-C₆ alkyl;
R² is C₁-C₄ alkyl;
or a pharmaceutically acceptable salt thereof, for use as inhibitor of RAS protein activation, for treatment and/or prophylaxis of diseases, wherein RAS-signalling is involved.

2. The compound of the formula (I) according to claim 1, 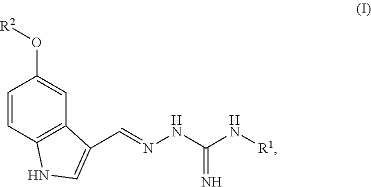
wherein
R¹ is C₁-C₆ alkyl;
R² is C₁-C₄ alkyl;
or a pharmaceutically acceptable salt thereof, for use as inhibitor of RAS protein activation.

3. The compound of formula (I) or a pharmaceutically acceptable salt thereof, according to claim 1 or 2, for use as inhibitor of RAS protein activation for treatment and/or prophylaxis of proliferative disorders, inflammatory diseases and/or genetic disorders, wherein RAS-signaling is involved.

4. The compound of formula (I), or a pharmaceutically acceptable salt thereof, according to claims 1 to 3, for use as inhibitor of RAS protein activation for treatment and/or prophylaxis of proliferative disorders, wherein KRAS12 A, KRAS G12V, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved.

5. The compound of formula (I), or a pharmaceutically acceptable salt thereof, according to claims 1 to 4, for use as inhibitor of RAS protein activation for treatment and/or prophylaxis of proliferative disorders and/or inflammatory diseases, which are resistant to treatment with RAS mutation specific inhibitors different from compounds of formula (I).

6. The compound of formula (I), or a pharmaceutically acceptable salt thereof, according to claim 5, wherein the resistance results from a secondary mutation, in particular results from a secondary mutation, wherein KRAS G12C/Y96D, KRAS G12C/Y96C and/or KRAS G12C/Y96S is involved.

7. The compound formula (I) according to claims 1 to 6, wherein
R¹ is C₃-C₆ alkyl;
R² is C₁-C₂ alkyl;
or a pharmaceutically acceptable salt thereof, for use as inhibitor of RAS protein activation, preferably for use as inhibitor of RAS protein activation, for treatment and/or prophylaxis, wherein RAS-signalling is involved, in particular for use as inhibitor of RAS protein activation for treatment and/or prophylaxis of proliferative disorders, inflammatory diseases and/or genetic disorders, wherein RAS-signaling is involved, especially wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved.

8. The compound of formula (I) according to any of the preceding claims, which is compound A 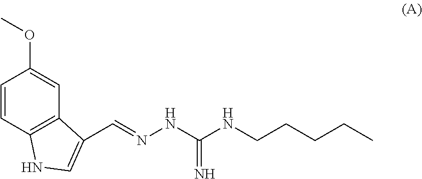 [SMILES: CCCCCNC(=N)N/N=C/c1c[nH]c2ccc(OC)cc12]
or a pharmaceutically acceptable salt thereof, for use as inhibitor of RAS protein activation.

9. The compound of formula (I) according to any of claims 1 to 8, which is compound A or a pharmaceutically acceptable salt thereof, for use as inhibitor of RAS protein activation, for treatment and/or prophylaxis of diseases, wherein RAS-signalling is involved.

10. The compound of formula (I) according to any of claims 1 to 9, which is compound A or a pharmaceutically acceptable salt thereof, for use as inhibitor of RAS protein activation, for treatment and/or prophylaxis of proliferative disorders, inflammatory diseases, and/or genetic disorders, wherein RAS-signaling is involved, particular, wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved.

11. The compound of formula (I) according to any of claims 1 to 9, which is compound A or a pharmaceutically acceptable salt thereof, for use as inhibitor of RAS protein activation for treatment and/or prophylaxis of proliferative disorders and/or inflammatory diseases, which are resistant to treatment with RAS mutation specific inhibitors different from compounds of formula (I), preferably wherein the resistance results from a secondary mutation, in particular results from a secondary mutation, wherein KRAS G12C/Y96D, KRAS G12C/Y96C and/or KRAS G12C/Y96S is involved.

12. The compound of formula (I) according to any of the claims 1 to 7, which is compound B 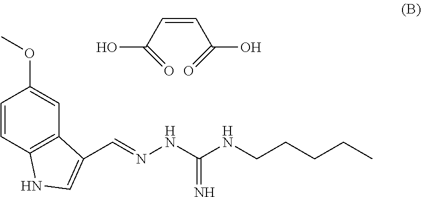 [SMILES: CCCCCNC(=N)N/N=C/c1c[nH]c2ccc(OC)cc12 ; O=C(O)/C=C\C(=O)O]
or a pharmaceutically acceptable salt thereof, for use as inhibitor of RAS protein activation.

13. The compound of formula (I) according to any of claim 1 to 7 or 12, which is compound B or a pharmaceutically acceptable salt thereof, for use as inhibitor of RAS protein activation, for treatment and/or prophylaxis of diseases, wherein RAS-signalling is involved.

14. The compound of formula (I) according to any of claim 1 to 7 or 12, which is compound B or a pharmaceutically acceptable salt thereof, for use as inhibitor of RAS protein activation, for treatment and/or prophylaxis of proliferative disorders, inflammatory diseases and/or genetic disorders, wherein RAS-signaling is involved, particular, wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved.

15. The compound of formula (I) according to any of claim 1 to 7 or 12, which is compound B or a pharmaceutically acceptable salt thereof, for use as inhibitor of RAS protein activation for treatment and/or prophylaxis of proliferative disorders and/or inflammatory disease, which are resistant to treatment with RAS mutation specific inhibitors different from compounds of formula (I), preferably wherein the resistance results from a secondary mutation, in particular results from a secondary mutation, wherein KRAS G12C/Y96D, KRAS G12C/Y96C and/or KRAS G12C/Y96S is involved.

16. A composition (1) comprising at least one compound of formula (I), in particular selected from compounds (A) or (B), as claimed in any one of claims 1 to 15, or a pharmaceutically acceptable salt, and RAS mutation specific inhibitors, in particular KRAS mutation specific inhibitors, especially sotorasib and/or adagrasib.

17. The compound of formula (I), in particular compound (A) or compound (B) according to any of claims 1 to 15 or a pharmaceutically acceptable salt thereof, or the composition (1) as claimed in claim16, for use in treating proliferative disorders, inflammatory diseases and/or genetic disorders wherein RAS-signaling is involved.

18. The compound of formula (I), in particular compound (A) or compound (B) according to any of claims 1 to 15 or a pharmaceutically acceptable salt thereof, or the composition (1) as claimed in claim 16, for use in treating proliferative disorders and/or inflammatory diseases, wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved.

19. The compound of formula (I), in particular compound (A) or compound (B) according to any of claims 1 to 15 or a pharmaceutically acceptable salt thereof, or the composition (1) as claimed in claim 16, for use in treating proliferative disorders, wherein the proliferative disorders is cancer, in particular the cancer is selected from prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, Haematological malignancies (including blood, bone marrow and lymph nodes) or testicular seminoma.

20. A pharmaceutical composition, comprising at least one compound of formula (I), in particular selected from compounds (A) or (B), as claimed in any one of claims 1 to 11, or a pharmaceutically acceptable salt thereof, or the composition (1) as claimed in claim 12, for the prophylaxis and/or treatment of proliferative disorders, inflammatory diseases and/or genetic disorders, wherein RAS-signaling is involved.

21. The pharmaceutical composition, comprising at least one compound of formula (I), in particular selected from compounds (A) or (B), as claimed in any one of claims 1 to 15, or a pharmaceutically acceptable salt thereof or the composition (1) as claimed in claim 16, for the prophylaxis and/or treatment of proliferative disorders and/or inflammatory diseases, wherein KRAS G12V, KRAS G12A, NRAS G12V, HRAS G12V, KRAS G12C, KRAS G12D, KRAS G12C/Y96D, KRAS G12C/Y96C, KRAS G12C/Y96S, KRAS G13C, KRAS G13D, KRASG13S, KRAS Q61H, KRAS Q61R or KRAS Q61K is involved.

22. The pharmaceutical composition, comprising at least one compound of formula (I), in particular selected from compounds (A), as claimed in any one of claims 1 to 11, or a pharmaceutically acceptable salt thereof or the composition (1) as claimed in claim 12, for use as inhibitor of RAS protein activation, for treatment and/or prophylaxis of proliferative disorders and/or inflammatory diseases, which are resistant to treatment with RAS mutation specific inhibitors different from compounds of formula (I), preferably wherein the resistance results from a secondary mutation, in particular results from a secondary mutation, wherein KRAS G12C/Y96D, KRAS G12C/Y96C and/or KRAS G12C/Y96S, is involved.

23. The pharmaceutical composition according to claims 20 to 22, comprising at least one compound of formula (I), in particular selected from compounds (A) and (B) or a pharmaceutically acceptable salt thereof, or the composition (1) as claimed in claim 12, and a pharmaceutically acceptable carrier.

24. The pharmaceutical composition according to claims 20 to 23, comprising additionally a further active substance, preferably selected from chemotherapeutic agents, radiotherapeutic agents, immuno-oncology agents and combinations thereof.

25. A method of inhibiting growth, proliferation, or metastasis of cancer cells in a subject in need thereof, said method comprising administering to the subject a therapeutically effective amount of at least one compound of formula (I), in particular selected from compounds (A) and (B), as defined in any one of claims 1 to 15, or a therapeutically acceptable salt thereof or the composition (1) as claimed in claim 16.

26. The method of claim 25, wherein the cancer is selected from prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, Haematological malignancies (including blood, bone marrow and lymph nodes) or testicular seminoma.

27. A method of inhibiting proliferation of a cell population sensitive towards inhibiting RAS activation in vitro or ex vivo, the method comprising contacting the cell population with at least one compound of the formula (I), in particular selected from compounds (A) and (B), as defined in any one of claims 1 to 16, or a therapeutically acceptable salt thereof or the composition (1) as claimed in claim 16.

28. A kit containing a formulation comprising: a) at least one compound of the formula (I), in particular selected from compounds (A) and (B), as defined in any one of claims 1 to 15, or a pharmaceutical composition as claimed in claim 16, comprising at least one compound of the formula (I), in particular selected from compounds (A) and (B), as defined in any one of claims 1 to 15, or a therapeutically acceptable salt thereof, or the composition (1) as claimed in claim 16, and a pharmaceutically acceptable carrier; and b) instructions for dosing of the pharmaceutical composition for the treatment of a disorder in which inhibition of RAS activation is effective in treating the disorder.
